(19) United States
(12) Patent Application Publication
(10) Pub. No.: US 2026/0151608 A1
(43) Pub. Date: Jun. 4, 2026

(54) MICRONEEDLE PATCH

(71) Applicant: DARWIN PRECISIONS CORPORATION, Hsinchu County (TW)

(72) Inventors: Yun-Pei Yang, Hsinchu County (TW); Jyun-Yi Yu, Hsinchu County (TW); Chung-Lin Lee, Hsinchu County (TW)

(21) Appl. No.: 19/283,280

(22) Filed: Jul. 29, 2025

(30) Foreign Application Priority Data
Dec. 3, 2024 (TW) 113146759

(51) Int. Cl. A61M 37/00

(52) U.S. Cl. CPC A61M 37/0015; A61M 2037/0023

(57) ABSTRACT

A microneedle patch includes a microneedle layer and an adhesive layer. The microneedle layer includes a plurality of microneedles and is composed of a biocompatible ingredient. The adhesive layer contains an effective ingredient, and the microneedle layer is disposed on the adhesive layer. The adhesive layer also includes a first region and a second region. The second region surrounds the first region, and the microneedle layer is located in the first region.

### FIELD OF THE INVENTION

The present invention relates to a field of transdermal drug delivery, and more particularly to a microneedle patch and composition thereof.

### BACKGROUND OF THE INVENTION

Microneedle patch (MNP) is a new type of transdermal drug delivery system (TDDS). The microneedles on the patch are quite short and do not touch nerves. Not only do they not cause a pain of subcutaneous injections, but they can also carry biologically active ingredients or drugs through the stratum corneum of the skin and enter the human body. MNP technology can be used in various fields such as aesthetic medicine, medicine, and preventive medicine.

### SUMMARY OF THE INVENTION

The present invention provides a microneedle patch which can provide a shorter duration of action and help accelerate an absorption of biologically active ingredients.

The present invention also provides a microneedle patch that can promote blood circulation in the skin.

To achieve one, part, or all of the above purposes or other purposes, one embodiment of the present invention provides a microneedle patch, including a microneedle layer and an adhesive layer. The microneedle layer includes a plurality of microneedles and is composed of biocompatible ingredients. The adhesive layer contains an effective ingredient, and the microneedle layer is disposed on the adhesive layer. The adhesive layer further includes a first region and a second region. The second region surrounds the first region, and the microneedle layer is located in the first region.

In one embodiment of the present invention, a ratio of an area of the second region to an area of the first region is between 0.05 and 8.

Because the adhesive layer of the present invention includes the first region and the second region, wherein the second region surrounds the first region, and the microneedle layer is located in the first region, the microneedle patch can release biologically active ingredients subcutaneously through the microneedle layer of the first region, and can also allow the effective ingredients to act on the skin through the adhesive layer, thereby achieving the effects of increasing subcutaneous vasodilation, promoting skin blood circulation, and inducing heat sensation. Increasing blood flow and rising temperature will, in turn, help accelerate the release of biologically active ingredients.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic cross-sectional view of a microneedle patch according to a first embodiment of the present invention;

FIG. 2 is a schematic top view of the microneedle patch according to the first embodiment of the present invention;

FIGS. 3A to 3D are schematic top views of microneedle patches according to a second embodiment of the present invention; and

FIG. 4 is a schematic diagram of hourly degradation rate of the microneedle layer of embodiments of the present invention and a comparative example over time.

### DETAILED DESCRIPTION OF PREFERRED EMBODIMENTS

The aforementioned and other technical contents, features and effects of the present invention will be clearly presented in the following detailed description of preferred embodiments with reference to the drawings. The direction terms mentioned in the following embodiments only refer to the directions of the attached drawings. Accordingly, the directional terms used are illustrative and not limiting of the present invention. In addition, terms such as “first” and “second” mentioned in this specification or the scope of the patent application are only used to name elements or to distinguish different embodiments or scopes, and are not used to limit the upper or lower limit on the number of elements.

The present invention provides a microneedle patch that can be used as a carrier of biologically active ingredients for transdermal absorption and transdermal drug delivery. The microneedle patch of the present invention can also promote subcutaneous release of the biologically active ingredients, thereby shortening action time and accelerating absorption.

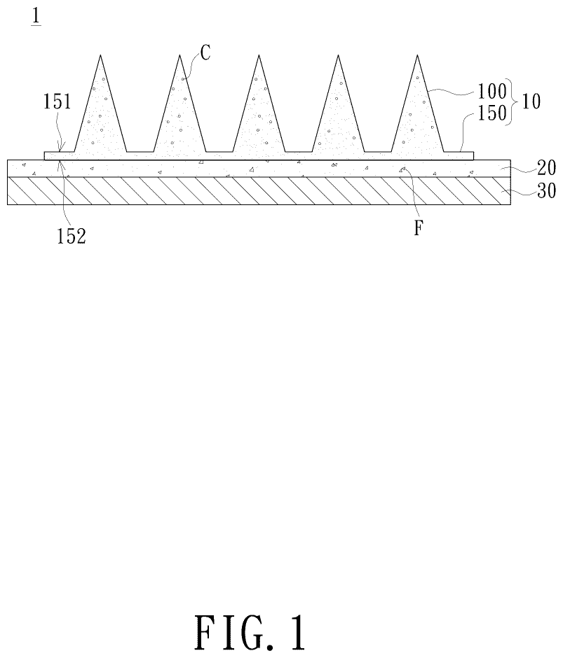

FIG. 1 is a schematic cross-sectional view of a microneedle patch according to an embodiment of the present invention. As shown in FIG. 1, the microneedle patch 1 includes a microneedle layer 10 and an adhesive layer 20. The microneedle layer 10 is disposed on the adhesive layer 20 and is composed of biocompatible ingredients. The “biocompatibility” of an ingredient means that a material, a compound, or a composition will not release toxic or harmful substances that cause local or systemic adverse reactions in a human body or other organisms, such as inflammatory reactions, immune reactions, and toxic reactions. In addition, in the embodiments of the present invention, the biocompatible ingredient is a material, a compound, or a composition that is degradable/dissolvable in the body. In a preferred embodiment of the present invention, the biocompatible ingredient includes one or more soluble polymer compounds, and the soluble polymer compounds impart solubility (or degradability) to the biocompatible ingredient. The soluble polymer compound can be a natural or artificially synthesized compound. For example, it can be maltodextrin, cyclodextrin, pullulan, carboxymethyl cellulose, methyl cellulose, hydroxyethyl cellulose, hydroxypropyl methylcellulose, polylactic acid, sodium alginate, hyaluronic acid, chitosan, polyvinylpyrrolidone or a combination thereof, or a polymer whose monomer is, for example, maltose, sucrose, lactose, trehalose or a combination thereof.

As shown in FIG. 1, the microneedle layer 10 is composed of biocompatible ingredients and also contains a biologically active ingredient C. In the present invention, the biologically active ingredient C means an ingredient that can produce biological effects on humans or other organisms, such as changing human physiological functions and biochemical metabolism. In the embodiment of the present invention, the microneedle layer 10 can achieve an effect of releasing the biologically active ingredient C therein by virtue of the solubility of the biologically active ingredient C. The biologically active ingredient C can be, for example, hyaluronic acid, nicotinamide, vitamin C, peptides, Centella asiatica, allantoin, dipotassium glycyrrhizinate, probiotics, yeast extract, or a combination thereof, but is not limited thereto. For example, the biologically active ingredient C may also be an ingredient having medicinal properties. In addition, the biologically active ingredient C of the embodiment of the present invention may be an ingredient suitable for taking effect in a short period of time and/or suitable for rapid absorption by the body. It should be noted that although the biologically active ingredient C is indicated in the figure, it is only used to indicate that the microneedle layer 10 contains the biologically active ingredient C. The biologically active ingredient C in the microneedle layer 10 is not necessarily visible.

As shown in FIG. 1, the microneedle layer 10 includes a plurality of microneedles 100, which can penetrate into the epidermis to release the biologically active ingredient C subcutaneously. The microneedle layer 10 further includes a base layer 150, wherein the plurality of microneedles 100 are spaced apart from each other and are disposed on the same side of the base layer 150. The diameter of the microneedles 100 becomes thicker as they are closer to the base layer 150. Specifically, the shape of the microneedles 100 can be, for example, a pyramid or a cone. The length of the microneedles 100 can be 100 to 1500 μm, such as 100 μm, 500 μm, 800 μm, 1000 μm, or 1500 μm. The base layer 150 has a first surface 151 and a second surface 152 opposite to each other. The microneedles 100 are disposed on the same surface (such as the first surface 151), and the other surface (such as the second surface 152) can be used to connect the microneedle layer 10 with the adhesive layer 20.

The adhesive layer 20 can include a layer composed of adhesive. The adhesive can be a natural or synthetic polymer compound, such as a rubber polymer, an acrylic acid polymer, a silicone, or a combination thereof, but is not limited thereto. The adhesive layer 20 preferably has affinity with the skin and can be sticky, but is not limited thereto. In addition, the adhesive layer 20 can also be a patch or a patch in a broad sense. When the adhesive layer 20 is in a form of a patch or a film, the patch or the film includes the aforementioned layer composed of adhesive. The adhesive layer 20 may further be in a form of gel or adhesive. In a preferred embodiment of the present invention, the adhesive layer 20 is a gel with silicone as a main component. As shown in FIG. 1, the microneedle patch 1 can further include a carrier layer 30. The carrier layer 30 is disposed on the adhesive layer 20 at the side opposite to the side connected to the microneedle layer 10, and its material can be, for example, natural or synthetic fabrics such as cloth, or natural or synthetic polymers such as film. The material of the carrier layer 30 can also be selected based on a number of functional requirements such as waterproof, breathable, antibacterial, odor, aesthetics, and hand feel.

In the embodiment of the present invention, the adhesive layer 20 contains an effective ingredient F. The effective ingredient F can be added into the aforementioned adhesive when preparing the adhesive layer 20. In a preferred embodiment of the present invention, the functions of the effective ingredient F are mainly to promote blood circulation, increase vasodilation, induce heat sensation, or a combination thereof. Based on the above effects, the microneedle patch 1 helps to increase the dilation of subcutaneous blood vessels and promote blood circulation in the skin. The increased blood flow can cause the local temperature of the skin to rise, causing a sense of heat. The increase in blood flow and the rise in temperature help to accelerate the degradation of the microneedles 100, thereby accelerating the release of the biologically active ingredient C. Any ingredients that can promote blood circulation, increase blood vessel dilation, cause skin temperature to rise, or induce a sense of heat can be used as the effective ingredient F of the embodiments of the present invention. Preferably, the effective ingredient F can be, for example, methylsalicylic acid, mint or extract thereof, capsaicin, vanillyl butyl ether, ginger or extract thereof, or a combination thereof. The present invention can also select or use in combination with various effective ingredients F based on other effects of the effective ingredients F, such as but not limited to detumescence and analgesia. It should be noted that although the effective ingredient F is indicated in the figure, it is only used to indicate that the adhesive layer 20 contains the effective ingredient F; the effective ingredient F in the adhesive layer 20 is not necessarily visible.

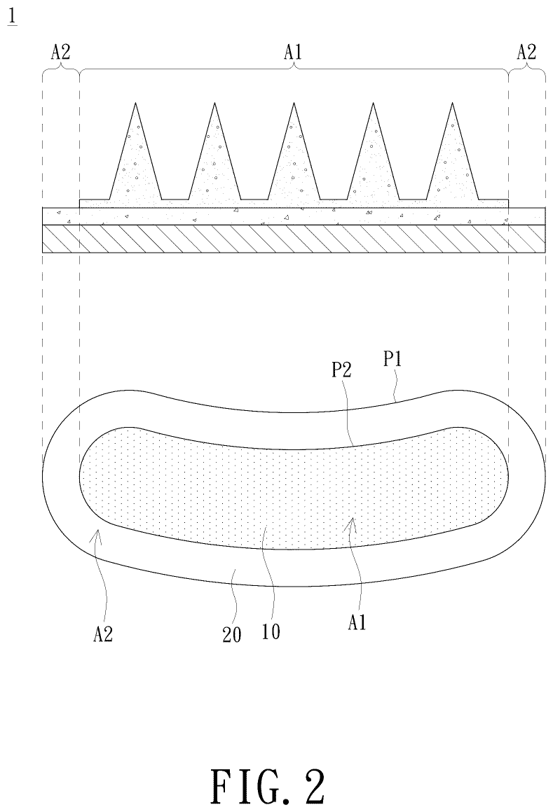

FIG. 2 is a schematic top view of the embodiment shown in FIG. 1. As shown in FIGS. 1 to 2, the adhesive layer 20 may have a specific shape and may be divided into different regions such as a first region A1 and a second region A2. The adhesive layer 20 further has the first region A1 and the second region A2 on a surface connected to the microneedle layer 10. In the embodiment of the present invention, the second region A2 surrounds the first region A1. For example, the second region A2 surrounding the first region A1 is an annular region when the first region A1 is a circular region. The microneedle layer 10 can be further located in the first region A1 of the adhesive layer 20. The shapes of the first region A1 and the second region A2 are not limited to standard geometric shapes, but may also be irregular shapes, such as the slightly curved oval shown in FIG. 2. On the other hand, an outer contour of the second region A2 can be equivalent to a shape of the adhesive layer 20 in the top view. For example, in the embodiment shown in FIG. 2, the adhesive layer 20 is a slightly curved oval shape which is equivalent to a shape of the outer contour P1 of the second region A2. An inner contour P2 of the second region A2 can be equivalent to a contour of the first region A1 when the first region A1 is adjacent to the second region A2, and the contour of the first region A1 is equivalent to a contour of the base layer 150 of the microneedle layer 10.

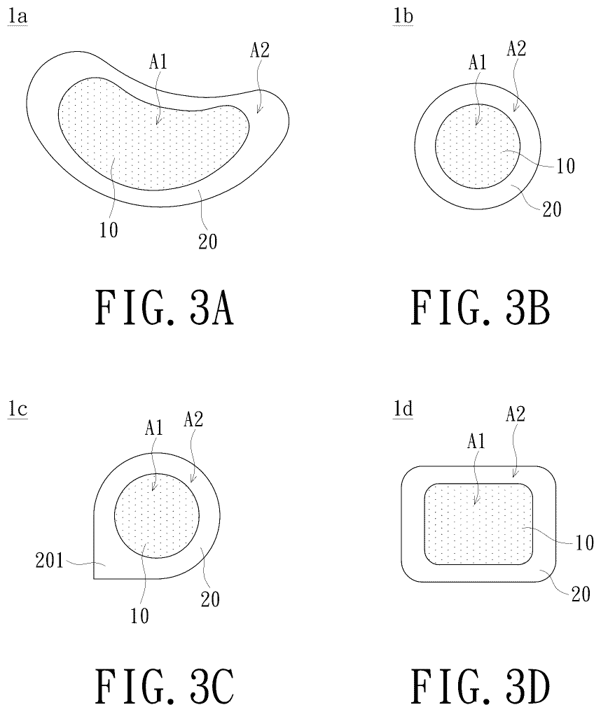

FIGS. 3A to 3D are schematic diagrams of another embodiment of microneedle patches of the present invention, wherein the first region A1 of the microneedle patch 1a in FIG. 3A is ear-shaped, the first regions A1 of the microneedle patch 1b in FIG. 3B and the microneedle patch 1c in FIG. 3C are circular, and the first region A1 of the microneedle patch 1d in FIG. 3D is a rectangle with rounded corners. In the embodiments shown in FIGS. 3A, 3B, and 3D, the shape of the first region A1 is substantially corresponding to the shape of the adhesive layer 20; that is, the adhesive layer 20 in FIG. 3A is also ear-shaped, the adhesive layer 20 in FIG. 3B is also circular, and the adhesive layer 20 in FIG. 3D is also rectangular, but the present invention is not limited thereto. In one embodiment of the present invention, the shape of the first region A1 can be different from the shape of the adhesive layer 20. For example, as shown in FIG. 3C, the first region A1 is circular and the adhesive layer 20 is of other shapes such as a teardrop shape. The design of the adhesive layer 20 and the first region A1 having corresponding or different shapes has the effect that, for example, when corresponding, the adhesive layer 20 can more completely cover the periphery of the microneedle layer 10, thereby ensuring that the skin around the microneedles 100 is affected as much as possible in the way of promoting blood circulation, increasing blood vessel dilation, causing the skin temperature to rise, and as a result, effectively accelerating the degradation of the microneedles 100. In other words, it is possible to prevent limiting the effect of the microneedles 100 due to the lack of the adhesive layer 20 around the microneedle layer 10. Furthermore, in response to usage requirements such as easy tearing and application for various shapes of different parts of the body, the adhesive layer 20 can also be designed to have a shape different from that of the first region A1. In a preferred embodiment of the present invention, the adhesive layer 20 may include only a first region A1 and a second region A2, and the sum of the area of the first region A1 and the area of the second region A2 is equal to the area of the adhesive layer 20.

As shown in FIGS. 1, 2, and 3A to 3D, the second region A2 corresponds to the portion of the adhesive layer 20 protruding outside the microneedle layer 10. Based on this design, on the one hand, the microneedle patch 1 can release the biologically active ingredient C subcutaneously through the microneedle layer 10 of the first region A1. On the other hand, because the second region A2 protrudes from the microneedle layer 10, the adhesive layer 20 can directly contact the skin. Thus, the microneedle patch 1 can allow the effective ingredient F to act on the skin through the adhesive layer 20, thereby achieving the effects of increasing subcutaneous vascular dilation, promoting skin blood circulation, warming the skin, and inducing heat sensation. The increase in blood flow and the rise in temperature help to accelerate the degradation of the microneedles 100, thereby accelerating the release of the biologically active ingredient C.

As shown in FIGS. 2 and 3A to 3D, the protrusion degree of the adhesive layer 20 is not limited to being uniform. For example, as shown in FIG. 3C, a portion 201 of the teardrop-shaped tip of the adhesive layer 20 protrudes more. The more the adhesive layer 20 protrudes, the more the degree of vasodilation, blood circulation and temperature rise can be expected to be increased. In the embodiment of the present invention, a ratio of an area of the second region A2 to an area of the first region A1 can roughly reflect the protrusion degree of the adhesive layer 20 and the proportion of the adhesive layer 20 to the microneedle layer 10 contacting the skin. The microneedle patch 1 is better to induce a more significant increase in blood flow and a rise in temperature when the ratio of the area of the second region A2 to the area of the first region A1 is larger, thereby accelerating the degradation of the microneedles 100 and the release of the biologically active ingredient C.

The ratio of the area of the second region A2 to the area of the first region A1 can be combined with a type and content of the effective ingredient F to obtain a desired degradation rate of the microneedles 100. Furthermore, the degradation rate of the microneedles 100 can be determined by a type and content of the biologically active ingredient C. For example, the effective ingredient F can be formulated according to the desired action time of the biologically active ingredient C in an organism, thereby allowing the microneedles 100 to have an appropriate degradation rate. In a preferred embodiment of the present invention, the ratio of the area of the second region A2 to the area of the first region A1 is between 0.05 and 8, for example, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 7.0, 7.5, or 8. The effective ingredient F can be one or more. Under the principle of safe dosage, the total amount of one or more effective ingredients F can account for 0.5 to 20 wt % of the adhesive layer 20, for example, 0.5 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %.

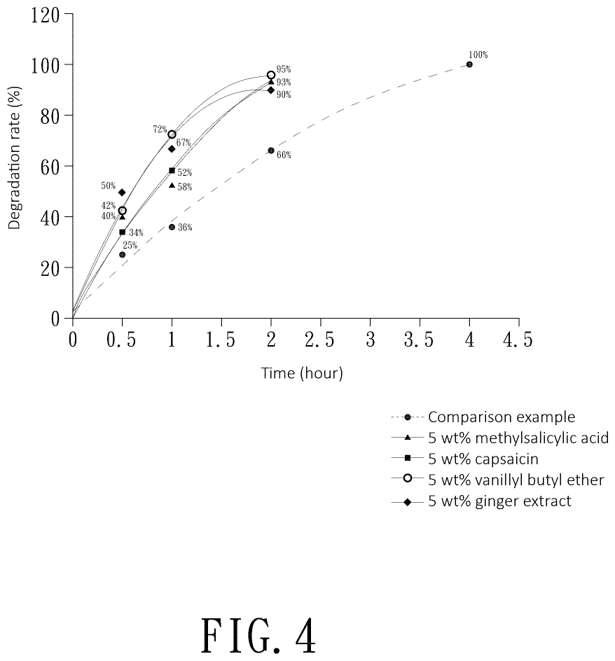

In a preferred embodiment of the present invention, the degradation rate of the microneedles 100 is complete (100%) degradation in less than four hours. In the present invention, the degradation rates of the microneedles of the microneedle patch 1 containing the effective ingredients F and the microneedle patch without the effective ingredients F are tested under the condition that the area ratio of the second region A2 to the first region A1 is the same. The test method can be to attach the microneedle patch 1 to the skin at room temperature, such as 25° C., and remove the microneedle patch 1 after, for example, 0.5, 1, 2, and 4 hours, to measure the lengths of the microneedles 100 therein. In a preferred embodiment of the present invention, repeated experiments are performed for different time groups, and the lengths of the microneedles 100 are measured and then the average value is calculated. The results are shown in Table 1 and FIG. 4.

TABLE 1

| | | Degradation rate |
|---|---|---|
| | | (degradation rate after 2 |
| Groups | | hours) |
| Comparison example (without the | | 66% |
| effective ingredients) | | |
| Embodiments | 1: 5 wt % | 93% |
| (containing | methylsalicylic acid | |
| the effective | 2: 5 wt % capsaicin | 93% |
| ingredients F) | 3: 5 wt % vanillyl butyl | 95% |
| | ether | |
| | 4: 5 wt % ginger extract | 90% |

Degradation rate calculation formula is: [(original length of microneedles−length of microneedles after n hours)/original length of microneedles]*100%.

Points in FIG. 4 represent the average degradation rates at each time of the embodiments 1, 2, 3, 4 and the comparative example. Linear regression analysis is then performed to obtain the degradation rate trend curves of the embodiments 1, 2, 3, 4 and the comparative example.

The approximate degradation rate can be obtained by dividing the degradation rate by the number of hours. For example, the degradation rate of the comparative example is approximately 33% per hour (66%/2=33%), and the degradation rate of the embodiment 4 (the effective ingredient F is 5 wt % ginger extract) is approximately 45% per hour (90%/2=45%), so the degradation rate of the embodiment 4 is greater than the degradation rate of the comparative example. It can be seen that the microneedle patches of the embodiments of the present invention can release the biologically active ingredient faster due to the increased degradation rate, which helps to shorten the action time and accelerate the absorption of the biologically active ingredient.

In summary, the microneedle patch 1 of the embodiment of the present invention can release the biologically active ingredient C subcutaneously through the microneedle layer 10, and can allow the effective ingredient F to act on the skin through the adhesive layer 20, thereby achieving the effects of increasing subcutaneous vascular dilation, promoting skin blood circulation, rising skin temperature, and inducing heat sensation. The increase in blood flow and the rise in temperature help to accelerate the degradation of the microneedles 100, thereby accelerating the release of the biologically active ingredient. The microneedle patch 1 of the embodiment of the present invention further has the effect of shortening the action time and accelerating absorption.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch, comprising:
a microneedle layer, comprising a plurality of microneedles and composed of a biocompatible ingredient; and
an adhesive layer, comprising an effective ingredient, wherein the microneedle layer is disposed on the adhesive layer;
wherein the adhesive layer comprises a first region and a second region, the second region surrounds the first region, and the microneedle layer is located in the first region.

2. The microneedle patch according to claim 1, wherein a ratio of an area of the second region to an area of the first region is between 0.05 and 8.

3. The microneedle patch according to claim 1, wherein the microneedle layer further comprises at least one biologically active ingredient and a base layer, the base layer has a first surface and a second surface opposite to the first surface, the plurality of microneedles is arranged on the first surface, and the microneedle layer is disposed in the first region with the second surface of the base layer.

4. The microneedle patch according to claim 1, wherein the effective ingredient is methylsalicylic acid, mint or extract thereof, capsaicin, vanillyl butyl ether, ginger or extract thereof, or a combination thereof.

5. The microneedle patch according to claim 4, wherein the adhesive layer is further composed of at least one polymer compound, and the effective ingredient accounts for 0.5 to 20 wt % of the adhesive layer.

6. The microneedle patch according to claim 1, wherein a sum of an area of the first region and an area of the second region is equal to an area of the adhesive layer.

7. The microneedle patch according to claim 1, wherein a shape of the adhesive layer corresponds to or is different from a shape of the first region.

8. The microneedle patch according to claim 7, wherein the shape of the adhesive layer is the same as the shape of the first region when the shape of the adhesive layer corresponds to the shape of the first region.
